(19) United States
(12) Patent Application Publication
(10) Pub. No.: US 2021/0330486 A1
(43) Pub. Date: Oct. 28, 2021

(54) ADJUSTABLE CERVICAL COLLAR

(71) Applicant: Tang Yong Kuang, Lucas, TX (US)

(72) Inventors: Tang Yong Kuang, Lucas, TX (US); Grace Tian-hui Kuang, Lucas, TX (US)

(73) Assignee: Tang Yong Kuang

(21) Appl. No.: 16/859,671

(22) Filed: Apr. 27, 2020

(51) Int. Cl. A61F 5/56; A61F 5/055

(52) U.S. Cl. CPC A61F 5/56; A61F 5/055

(57) ABSTRACT

An adjustable cervical collar, comprising, a curved top board having a width of approximately a size of a human chin, the curved top board having an arcuate inner edge capable of fitment around the front of a throat, the curved top board having a curved outer region distal to the throat, two upper columns couple-able to a lower portion of the curved top board, two lower columns slidably couple-able to the two upper columns and a curved bottom board detachably couple-able to the two lower columns.

### BACKGROUND

### Technical Field

The instant disclosure is related to a mechanism to insure an unobstructed airway during sleep and specifically an adjustable cervical collar.

### Background

The majority of the adult population snores at various intensities during sleep, those who snore louder may have obstructive sleep apnea (OSA) due to blockage of airflow from the collapsing of the upper airway. Based on the diagnostic criteria of apnea-hypopnea index (AHI)≥5 times/hour per the American Academy of Sleep Medicine (AASM) in 2012, an estimated 936 million people worldwide between the ages of 30 and 69 have OSA; among them an estimated 425 million people worldwide meet the requirement for moderate OSA with AHI≥15 times/hour that are usually recommended for treatment as it increases risk of cardiovascular comorbidities if left untreated.

At present, there is no easy treatment option for OSA. The most widely relied upon treatment method is to use continuous positive airway pressure treatment (CPAP) during sleep. Alternative treatment options include oral appliances, surgery and/or weight loss. CPAP and oral appliance treatment are uncomfortable, inconvenient, and have only a 50-60% compliance rate; especially when the CPAP pressure is high. When the CPAP pressure is high the mask is prone to air leaks and discomfort. There are multiple limitations related to a surgical treatment option, which include high cost, surgical risks and a relatively low effective cure rate of about 60%.

What is sought is another method of providing an unobstructed airway during sleep that does not rely upon a CPAP or surgery.

### SUMMARY

An example adjustable cervical collar, including at least one of, a curved top board having a width of approximately a size of a human chin, the curved top board having an arcuate inner edge capable of fitment around the front of a throat, the curved top board having a curved outer region distal to the throat, two upper columns couple-able to a lower portion of the curved top board, two lower columns slidably couple-able to the two upper columns and a curved bottom board detachably couple-able to the two lower columns.

### DESCRIPTION OF THE DRAWINGS

In the drawings:

FIG. 1 is a first example adjustable cervical collar in accordance with one embodiment of the disclosure;

FIG. 2 is an example adjustable cervical collar including a neck strap in accordance with one embodiment of the disclosure;

FIG. 3 is an example system chin strap and neck strap in accordance with one embodiment of the disclosure;

FIG. 4 depicts example neck straps in accordance with embodiments of the disclosure:

FIG. 5 depicts example neck strap clips in accordance with embodiments of the disclosure;

FIG. 6 is a second example adjustable cervical collar in accordance with one embodiment of the disclosure;

FIG. 7 is a third example adjustable cervical collar in accordance with one embodiment of the disclosure; and

FIG. 8 depicts a cervicomental angle between the submental plane to the cervical plane.

### DETAILED DESCRIPTION OF THE INVENTION

The embodiments listed below are written only to illustrate the applications of this apparatus and method, not to limit the scope. The equivalent form of modifications towards this apparatus and method shall be categorized as within the scope the claims.

Certain terms are used throughout the following description and claims to refer to particular system components. As one skilled in the art will appreciate, different companies may refer to a component and/or method by different names. This document does not intend to distinguish between components and/or methods that differ in name but not in function.

In the following discussion and in the claims, the terms “including” and “comprising” are used in an open-ended fashion, and thus may be interpreted to mean “including, but not limited to . . . .” Also, the term “couple” or “couples” is intended to mean either an indirect or direct connection. Thus, if a first device couples to a second device that connection may be through a direct connection or through an indirect connection via other devices and connections.

During cardiopulmonary resuscitation (CPR), the head is tilted to a chin-lift position to keep the upper airway passage open. Based on this observation, the adjustable cervical collar supports of the neck and tilt of the head so that users can maintain a head-tilt chin-lift position during sleep to keep their upper airway open.

The device may keep the upper airway open more effectively, which may relieve snoring and obstructive sleep apnea when lying on the back or side positions. If CPAP therapy is used simultaneously it may reduce the CPAP pressure requirement and improve treatment compliance with less leakage and discomfort at CPAP mask. The height of cervical collar may be adjusted to an optimal level for the user, thus increasing comfort and effectiveness. The collar may also be used during long-distance travel or other situations when a user's neck may benefit from support such as dozing while sitting.

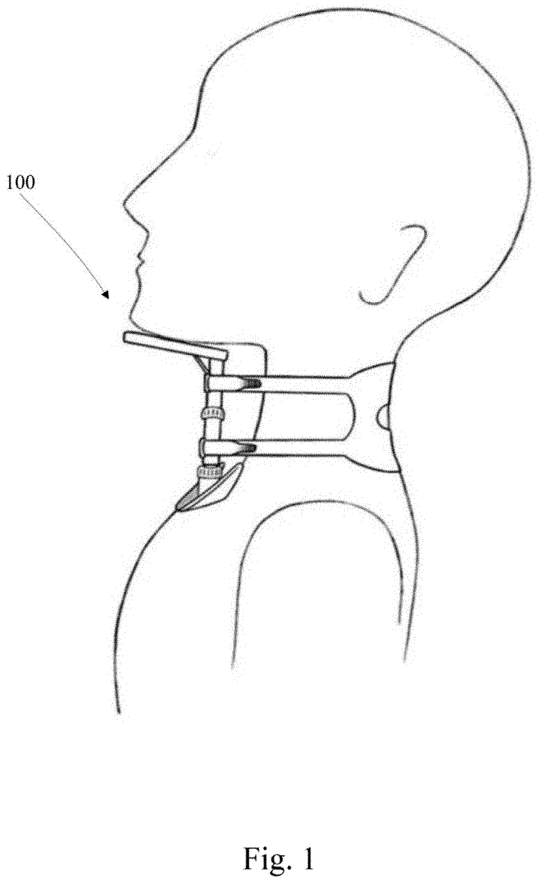

FIG. 1 depicts an example adjustable cervical collar 100 on a user. The device depicts a neck strap holding the adjustable cervical collar in place to prevent the chin of the user from lowering toward the chest, resulting in an obstructed airway.

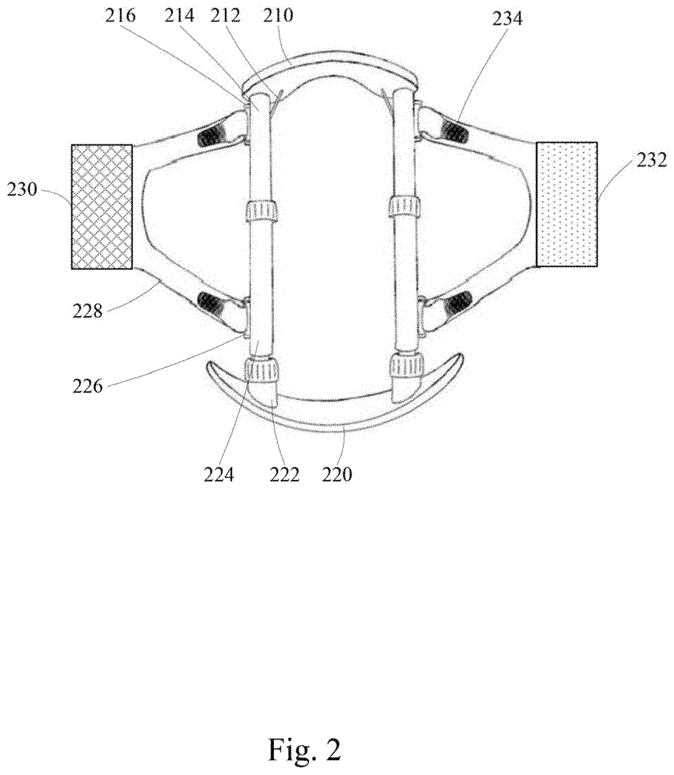

FIG. 2 shows an example adjustable cervical collar and neck strap. A curved top board 210 may have a width approximately a size of a human chin, the curved top board 210 may have a throat shape cutout approximately a size of a human throat on the side proximate the user's throat. The curved top board has an arcuate inner edge capable of fitment around the front of the throat. The curved top board has a curved outer portion distal to the user's throat. The collar may include a support rib 212 connected to the curved top board and the upper columns 214. The two upper columns 214 may be couple-able to a lower portion of the curved top board 210. An upper distribution ring 216 may be connected to the two upper columns 214. Two lower columns 224 may be slidably couple-able to the two upper columns 214. A curved bottom board 220 may be detachably couple-able to the two lower columns 224. The two upper columns 214 may be complementary to the two lower columns 224. A lower distribution ring 226 may be connected to the two lower columns 224. In this example a neck strap 228 is coupled to the upper distribution ring 216 and the lower distribution ring 226. In this example, the neck strap 228 includes a hook 230 and loop 232 connector. The adjustable cervical collar may be manufactured of hypoallergenic plastic or silicon.

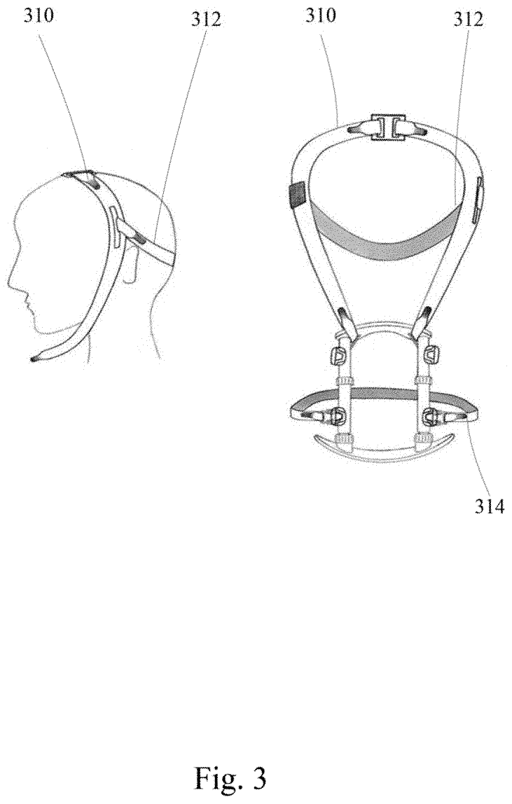

FIG. 3 depicts examples of chin straps 310, connected to head straps 312. The chin straps 310 may be coupled to the support rib (FIG. 2, 212). A neck strap 314 may be connected to the lower distribution ring (FIG. 2, 226). The chin straps, head straps and neck straps may be made of a hypoallergenic material.

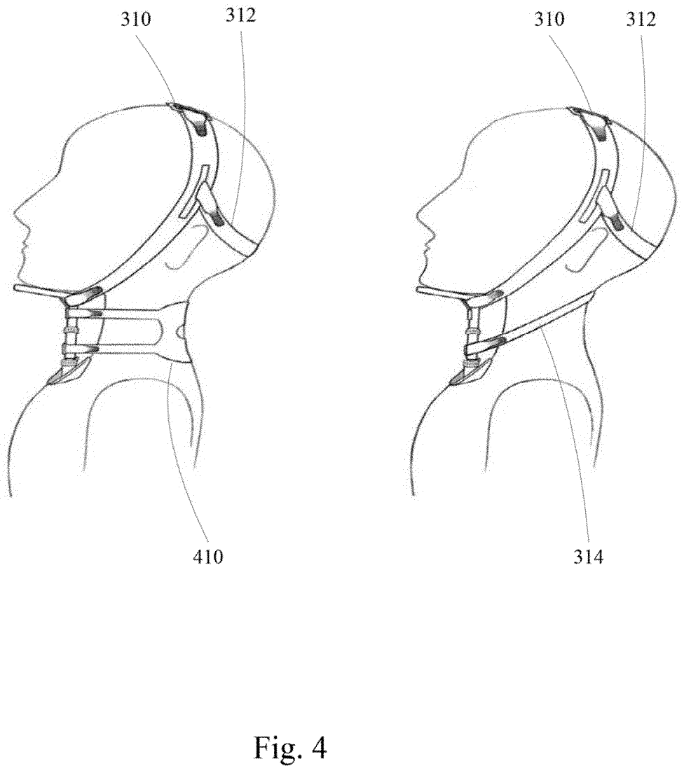

FIG. 4 depicts two examples of strapping the adjustable cervical collar to a user. In both examples the chin straps 310 are connected to head straps 312. In the left hand example, a neck strap 410 is coupled to both the upper distribution ring (FIG. 2, 216) and the lower distribution ring (FIG. 2, 226). In the right hand side example, a neck strap 314 is coupled to the lower distribution ring (FIG. 2, 226).

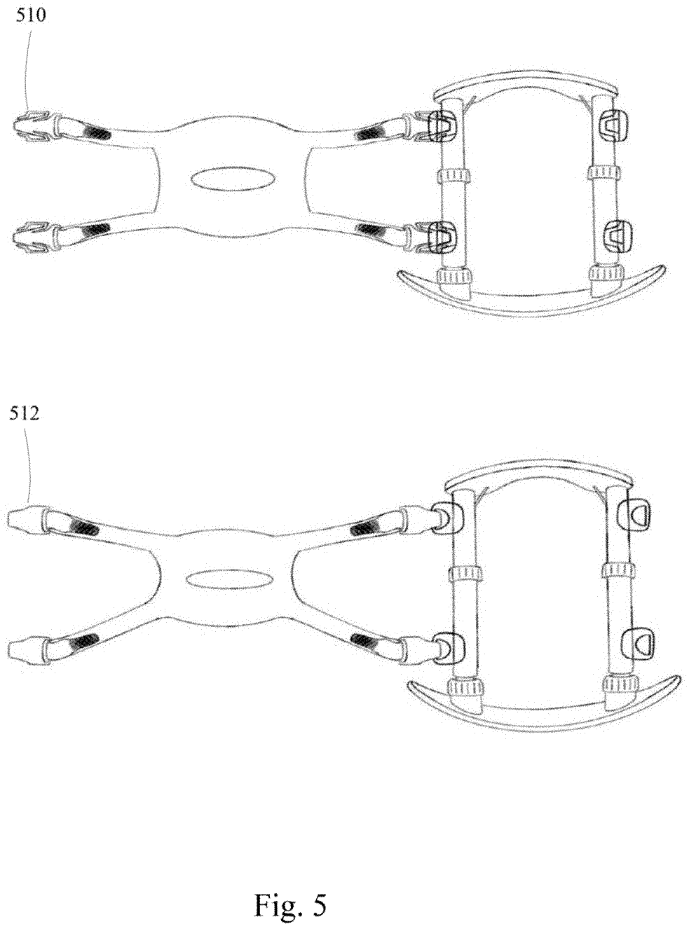

FIG. 5 depicts example neck strap clips 510 and 512. The neck strap clips may be plastic, metal or the like.

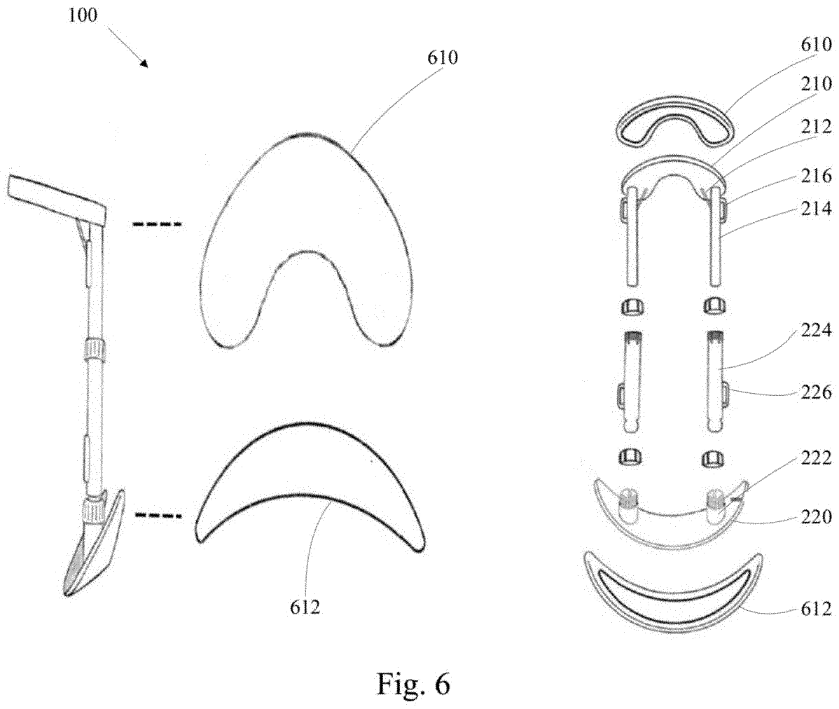

FIG. 6 depicts an example adjustable cervical collar having a top pad 610 detachably coupled to the curved top board 210 and a bottom pad 612 detachably coupled to the curved bottom board 220. The top pad 610 and the bottom pad 612 may be comprised of non-allergenic materials. The curved top board 210 may have a width approximately a size of a human chin, the curved top board 210 has a throat shape cutout approximately a size of a human throat on the side proximate the user's throat. The curved top board has an arcuate inner edge capable of fitment around the front of the throat. The curved top board has a curved outer region distal to the user's throat. The collar may include a support rib 212 connected to the curved top board and the upper columns 214. The two upper columns 214 may be couple-able to a lower portion of the curved top board 210. An upper distribution ring 216 may be connected to the two upper columns 214. Two lower columns 224 may be slidably couple-able to the two upper columns 214. A curved bottom board 220 may be detachably couple-able to the two lower columns 224. The two upper columns 214 may be complementary to the two lower columns 224. A lower distribution ring 226 may be connected to the two lower columns 224. In this example a neck strap 228 is coupled to the upper distribution ring 216 and the lower distribution ring 226. In this example, the neck strap 228 includes a hook 230 and loop 232 connector.

In this example to assemble the adjustable cervical collar, the round head at the bottom of the two lower columns 224 may be inserted into the joint socket 222 on the curved bottom board 220 and secured with the lower nut. This may result in a rotatable joint so that the bottom board may be tilted to closely contact the user's chest. Four slits equally distributed at each side of the thread on the joint socket 222 may allow it to be squeezed tightly by the nut to hold it in place. The upper columns 214 may be inserted into the two lower columns 224 and the height of the cervical collar adjusted and secured with the upper nut. The top pad 610 and bottom pad 612 may be cushions made of foam to cover the curved top board 210 and to the curved bottom board 220 for comfort. The cervical collar may be secured at front of a user's neck with a neck strap and optional chin strip and head strap.

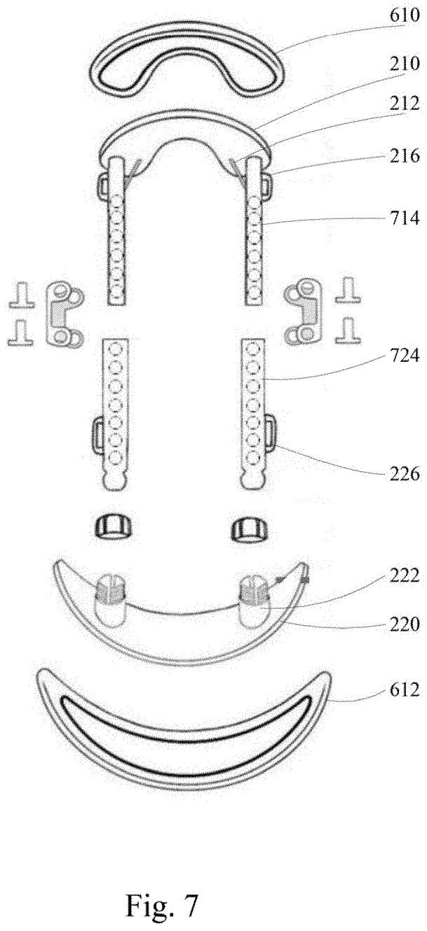

FIG. 7 depicts another example adjustable cervical collar having a top pad 610 coupled to the curved top board 210 and a bottom pad 612 couple to the curved bottom board 220. The curved top board 210 may have a width approximately a size of a human chin, the curved top board 210 has a throat shape cutout approximately a size of a human throat on the side proximate the user's throat. The curved top board has an arcuate inner edge capable of fitment around the front of the throat. The curved top board has a curved outer region distal to the user's throat. The collar may include a support rib 212 connected to the curved top board and the upper columns 714 having retaining holes therein. The upper columns 714 may be couple-able to a lower portion of the curved top board 210. An upper distribution ring 216 may be connected to the two upper columns 714. Two lower columns 724 may be pinned to the two upper columns 714. A curved bottom board 220 may be detachably couple-able to the two lower columns 724. The two upper columns 714 may be complementary to the two lower columns 724. A lower distribution ring 226 may be connected to the two lower columns 724.

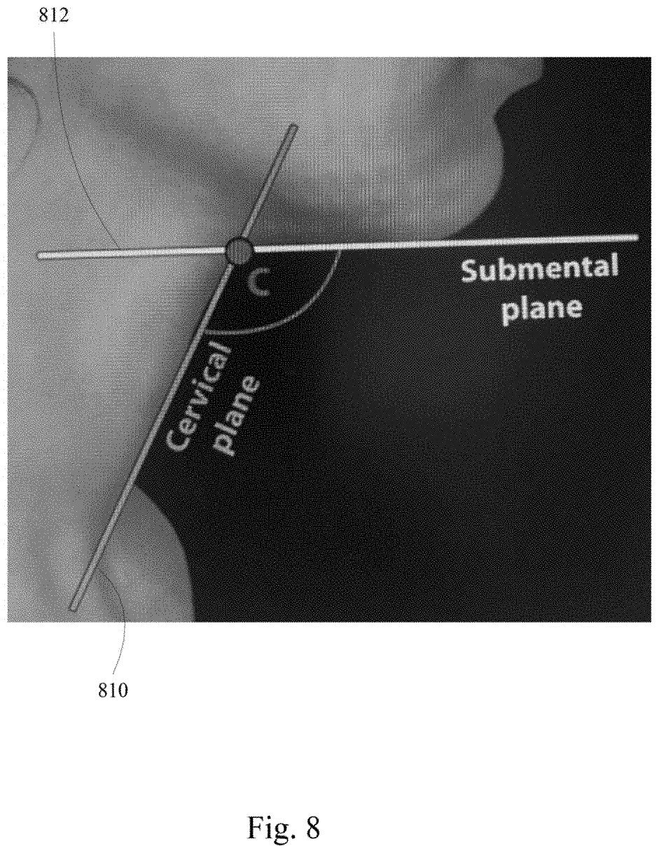

FIG. 8 depicts an example cervicomental angle between the cervical plane 810 and the submental plane 812 of a user. The adjustable cervical collar may approximate a cardiopulmonary resuscitation chin lift during use wherein the curved top board forms approximately 100 to approximately 120 degree angle with the two upper columns. This 100 to 120 degree angle may be the angle between the cervical plane 810 and the submental plane 812.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention. The predicate words “configured to”. “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code may be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

The word “example” is used herein to mean “serving as an example or illustration.” Any aspect or design described herein as “example” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

References to “one embodiment,” “an embodiment,” “some embodiments,” “various embodiments”, or the like indicate that a particular element or characteristic is included in at least one embodiment of the invention. Although the phrases may appear in various places, the phrases do not necessarily refer to the same embodiment. In conjunction with the present disclosure, those skilled in the art will be able to design and incorporate any one of the variety of mechanisms suitable for accomplishing the above described functionalities.

It is to be understood that the disclosure teaches just one example of the illustrative embodiment and that many variations of the invention can easily be devised by those skilled in the art after reading this disclosure and that the scope of then present invention is to be determined by the following claims.

1. An adjustable cervical collar, comprising:
a curved top board capable of receiving and supporting a human chin,
the curved top board having an arcuate inner edge capable of fitment around a front of a throat,
the curved top board having a curved outer region distal to and capable of fitment around the throat;
two upper columns couple-able to a lower portion of the curved top board;
two lower columns slidably couple-able to the two upper columns resulting in a rotatable joint; and
a curved bottom board detachably couple-able to the two lower columns,
wherein the collar supports a neck and a tilt of a head to maintain a head-tilt chin-lift position during sleep to keep an upper airway open so as to relieve obstructive sleep apnea when lying on a back or a side position during sleep.

2. The adjustable cervical collar of claim 1, further comprising at least one support rib connected to the curved top board and at least one of the two upper columns.

3. The adjustable cervical collar of claim 2, further comprising a chin strap couple-able to the at least one support rib.

4. The adjustable cervical collar of claim 3, further comprising a head strap couple-able to the chin strap.

5. The adjustable cervical collar of claim 1, further comprising at least one upper distribution ring connected to at least one of the two upper columns.

6. The adjustable cervical collar of claim 5, further comprising a neck strap couple-able to the at least one upper distribution ring.

7. The adjustable cervical collar of claim 6, further comprising a hook and loop coupling coupled to the neck strap.

8. The adjustable cervical collar of claim 6, further comprising at least one clip coupled to the neck strap.

9. The adjustable cervical collar of claim 1, wherein at least one of the two upper columns is complementary to at least one of the two lower columns.

10. The adjustable cervical collar of claim 1, further comprising a top pad detachably couple-able to the curved top board.

11. The adjustable cervical collar of claim 1, further comprising a bottom pad detachably couple-able to the curved bottom board.

12. The adjustable cervical collar of claim 1, further comprising at least one lower distribution ring connected to at least one of the two lower columns.

13. The adjustable cervical collar of claim 1, wherein at least one of the two lower columns has a slit coupling.

14. The adjustable cervical collar of claim 1, wherein the adjustable cervical collar approximates a cardiopulmonary resuscitation chin lift during use.

15. The adjustable cervical collar of claim 1, wherein the curved top board forms an angle of 100 to 120 degrees with the two upper columns.

16. The adjustable cervical collar of claim 1, wherein the curved top board is formed of a non-allergenic material.

17. The adjustable cervical collar of claim 1, wherein the two lower columns are pinned to the two upper columns.
